(19) United States
(12) Patent Application Publication
(10) Pub. No.: US 2026/0096786 A1
(43) Pub. Date: Apr. 9, 2026

(54) SYSTEMS AND METHODS FOR AUTOMATIC STATE ESTIMATION OF A CURRENT IMAGING EXAM USING USER ACTIONS ON A CONSOLE SCREEN

(71) Applicant: KONINKLIJKE PHILIPS N.V., EINDHOVEN (NL)

(72) Inventors: SIVA CHAITANYA CHADUVULA, MALDEN, MA (US); EKIN KOKER, CAMBRIDGE, MA (US); OLGA STAROBINETS, NEWTON, MA (US); RANJITH NAVEEN TELLIS, TEWKSBURY, MA (US); SANDEEP MADHUKAR DALAL, WINCHESTER, MA (US); THOMAS ERIK AMTHOR, HAMBURG (DE); YUECHEN QIAN, LEXINGTON, MA (US)

(21) Appl. No.: 19/112,526

(22) PCT Filed: Sep. 19, 2023
(86) PCT No.: PCT/EP2023/075808

§ 371 Date: Mar. 17, 2025

Related U.S. Application Data
(60) Provisional application No. 63/408,133, filed on Sep. 20, 2022.

(51) Int. Cl. A61B 5/00; G06T 7/00; G06T 7/20; G06V 10/75; G06V 20/40; G16H 40/67

(52) U.S. Cl. CPC A61B 5/747; A61B 5/0077; A61B 5/7282; A61B 5/742; A61B 5/746; G06T 7/0012; G06T 7/20; G06V 10/75; G06V 20/44; G16H 40/67; G06T 2200/24; G06T 2207/10016; G06T 2207/30016; G06V 2201/03

(57) ABSTRACT

A non-transitory computer readable medium (26s) stores instructions executable by at least one electronic processor (14s) to perform a method (100) of providing assistance during a medical imaging examination performed using a medical imaging device (2). The method includes acquiring video (17) of the medical imaging examination; determining, using a state machine (40) implemented in the at least one electronic processor, a current state of an imaging examination from the acquired video; and displaying an indication (46) of the determined current state of the imaging examination on an electronic processing device (8).

The following relates generally to the imaging arts, remote imaging assistance arts, remote imaging examination monitoring arts, and related arts.

### BACKGROUND

Medical imaging, such as computed tomography (CT) imaging, magnetic resonance imaging (MRI), positron emission tomography (PET) imaging, fluoroscopy imaging, and so forth, is a critical component of providing medical care, and is used in a wide range of medical fields, such as cardiology, oncology, neurology, orthopedics, to name a few. The operator of the medical imaging device used to acquire the medical images is typically a trained technologists, while interpretation of the medical images is often handled by a medical specialist such as a radiologist. Interpretation of radiology reports or findings by the radiologist can be handled by the patient's general practitioner (GP) physician or a medical specialist such as a cardiologist, oncologist, orthopedic surgeon, or so forth.

Currently, diagnostic imaging is in high demand. As the world population ages, the demand for quick, safe, high quality imaging will only continue to grow, putting further pressure on imaging centers and their staff Under such conditions, errors are unavoidable, but can be often costly. One approach for imaging centers to boost efficiency and grow operations at no extra labor costs is through a radiology operations command center (ROCC) system. Radiology operations command centers enable teams to work across the entire network of imaging sites, providing their expertise as needed and remotely assisting less experienced technologists in carrying out high quality scans. Remote technologists or experts can monitor the local operators of scanning procedures through cameras installed in the scanning areas (or from other sources, such as sensors (including radar sensors), console video feeds, microphones connected to Internet of Things (IoT) devices, and so forth. In addition, these sources can be supplemented by other data sources like Health-Level 7 (HL7), Digital Imaging and Communications in Medicine (DICOM), Electronic Health Record (EHR) databases, and so forth.

The remote technologist (i.e. “super-tech;” also referred to herein as an “expert tech” or a remote expert)) is expected to be concurrently assigned to assist a number of different imaging bays at different sites that may be spread out across different cities or different states. In practice, however, the super-tech can only be paying attention to a single imaging bay at any given time. The super-tech will typically be assisting local technologists who actively call for super-tech support. However, situations may arise in which the super-tech's assistance would be beneficial, but the local technologist is unaware of the need for super-tech assistance, or chooses not to call for such assistance.

During image acquisition using MR or CT, the users (or technologists) can perform wide range of activities including planning the scans, review images from the current/past exams, add/repeat sequences based on the patient and current exam's context. The asynchronous nature of these activities makes it harder to determine the current state of the exam. A lack of exam's current state inhibits expert user to offer help for the remote/local technologist pro-actively.

The following discloses certain improvements to overcome these problems and others.

### SUMMARY

In one aspect, a non-transitory computer readable medium stores instructions executable by at least one electronic processor to perform a method of providing assistance during a medical imaging examination performed using a medical imaging device. The method includes acquiring video of the medical imaging examination; determining, using a state machine implemented in the at least one electronic processor, a current state of an imaging examination from the acquired video; and displaying an indication of the determined current state of the imaging examination on an electronic processing device.

In another aspect, a method of providing assistance during a medical imaging examination performed using a medical imaging device includes acquiring video of the medical imaging examination; tracking progress of the medical imaging examination using a state machine representing a workflow of the medical imaging examination, the progress being tracked based at least on matching information extracted from the acquired video with state information of states of the state machine; and performing an assistive action to provide assistance during the medical imaging examination based on the tracked progress of the medical imaging examination.

In another aspect, a non-transitory computer readable medium stores instructions executable by at least one electronic processor to perform a method of providing assistance during a medical imaging examination performed using a medical imaging device. The method includes acquiring video of the medical imaging examination; determining, using a state machine implemented in the at least one electronic processor, a current state of an imaging examination from the acquired video; determining an event of the medical imaging examination that triggered the transition from the current state of the medical imaging examination to a next state of the medical imaging examination based on a state transition of the state machine from the current state to the next state; and displaying an indication of the determined current state of the imaging examination on an electronic processing device.

One advantage resides in providing alerts to a remote expert of events occurring during a procedure operable by a local operator.

Another advantage resides in determining a state of an imaging examination.

Another advantage resides in providing an automatic method of capturing a state of an imaging examination based on actions performed by a user on an imaging device console.

Another advantage resides in providing for tracking progress of the medical imaging examination using a state machine representing a workflow of the medical imaging examination, thereby enabling fine-grained detection of complex events that may occur during a given medical imaging examination.

Another advantage resides in providing assistance to a local operator performing a medical imaging examination based on such tracked progress.

Another advantage resides in collecting data on a performance of a local operator performing the medical imaging examination based on such tracked progress.

Another advantage resides in analyzing a timeseries of exam states along with patient/exam characteristics to provide a reliable quantification of technologist expertise that enables an operational manager to not only use their pool of technologists efficiently but also adhere to the standard practices followed at their respective medical facilities.

Another advantage resides in improving efficiency of handling patients during imaging examinations.

Another advantage resides in a technologist to check for adherence to policies for imaging examinations while an imaging examination is taking place.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative apparatus for providing remote assistance in accordance with the present disclosure.

FIG. 2 show an example flow chart of operations suitably performed by the apparatus of FIG. 1.

FIG. 3 diagrammatically shows an illustrative simplified state machine representing a medical imaging examination workflow.

### DETAILED DESCRIPTION

The following relates to a ROCC framework, developed to enable communication between local imaging technologists performing imaging examinations and remote experts, in which a state machine is used to determine the current state of the examination.

The state machine may be constructed manually, and is typically specific to the particular imaging device make and model (or model series, as may be appropriate), software version (as may be appropriate), and the particular imaging workflow (e.g., brain scan, full body scan, et cetera). Manual construction of the state machine is feasible since there are a relatively small number of states in any given workflow, and the computerized workflow implemented by the imaging device controller transitions between well-defined states with corresponding user interface (UI) dialogs and/or UI dialog content. As the state will be assessed based on information gleaned from the scraped controller screen, state transitions are identified by changes in the displayed content, such as switching from one UI dialog to another, detecting the opening of a pop-up window, identifying newly displayed text, and/or so forth. Each state is characterized by a vector of values for state variables.

During an imaging session, the scraped screen is monitored. An initial state can be identified by a known startup UI dialog, for example via which the imaging technician enters patient and scan information. From the initial state, transitions between states of the state machine representing the medical imaging examination workflow are identified from changes in the scraped screen as just described. To improve efficiency, the various pipelines for detecting display content changes corresponding to examination state changes can be filtered, for example applying OCR only to text that has changed.

The resulting real-time tracking of the progress of the medical imaging examination provides a rich source of information that can be used in real-time during the examination to guide ROCC operations. For example, if the examination remains in the patient loading state for an inordinate time, then this might trigger an alert to a remote expert to contact the imaging technician to see if assistance is needed. As another example, if the medical imaging examination makes a “regressive” transition through the state machine, for example from a state representing image acquisition to a state representing patient positioning, this may indicate an event such as a problem arising in the medical imaging examination. Such a detected event can be used to trigger an assistive action to assist the local operator, such as establishing a natural communication pathway (e.g. video call) between the local operator performing the medical imaging examination and a remote expert, or automatically providing textual, graphical, video, and/or multimedia guidance respective to the determined event.

The real-time tracked progress of the medical imaging examination in terms of states traversed, time duration in each state, transitions between states, or so forth can be recorded for most or all imaging examinations performed by a radiology department or other entity, and can be stored and subsequently mined for various purposes, such as to detect performance deficiencies of a specific imaging technician to identify areas where that technician requires further training, to identify areas where the radiology laboratory workflow is inefficient (for example, if a torso imaging examination frequently loops back to re-acquisition of an image that requires a patient breath-hold then this may indicate that the workflow for instructing the patient on the breath-hold should be reviewed), and so forth.

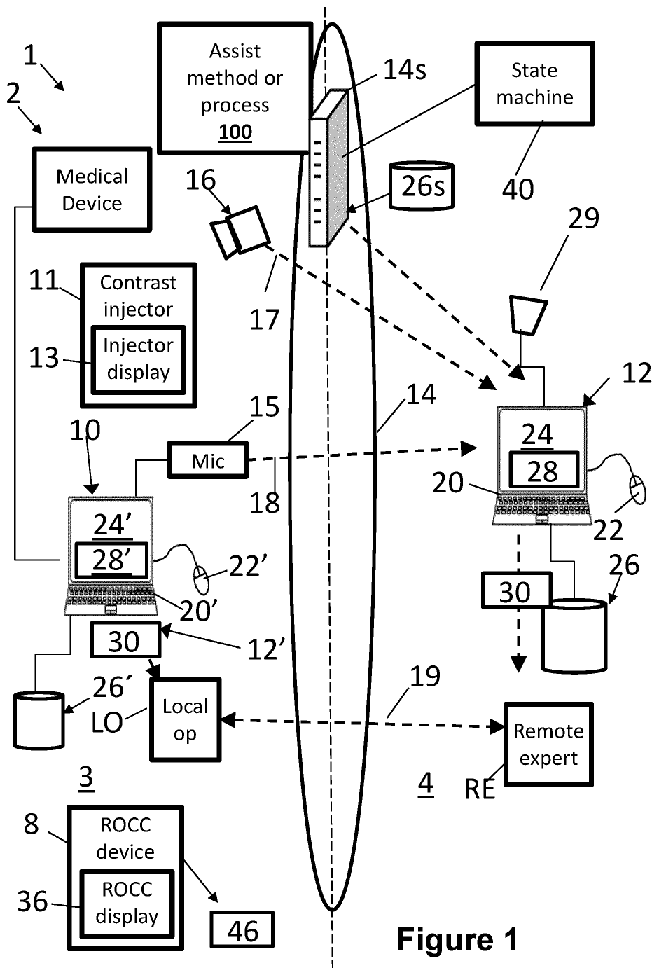

With reference to FIG. 1, an apparatus 1 for providing assistance from a remote medical imaging expert RE (or supertech) to a local technologist operator LO is shown. As shown in FIG. 1, the local operator LO, who operates a medical imaging device (also referred to as an image acquisition device, imaging device, and so forth) 2, is located in a medical imaging device bay 3, and the remote expert RE is disposed in a remote service location or center 4. It should be noted that the “remote expert” RE may not necessarily directly operate the medical imaging device 2, but rather provides assistance to the local operator LO in the form of advice, guidance, instructions, or the like. The remote location 4 can be a remote service center, a radiologist's office, a radiology department, and so forth. The remote location 4 may be in the same building as the medical imaging device bay 3 (this may, for example, in the case of a “remote operator or expert” RE who is a radiologist tasked with peri-examination image review), but more typically the remote service center 4 and the medical imaging device bay 3 are in different buildings, and indeed may be located in different cities, different countries, and/or different continents. In general, the remote location 4 is remote from the imaging device bay 3 in the sense that the remote expert RE cannot directly visually observe the imaging device 2 in the imaging device bay 3 (hence optionally providing a video feed as described further herein).

The image acquisition device 2 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The imaging device 2 may also be a hybrid imaging device such as a PET/CT or SPECT/CT imaging system. While a single image acquisition device 2 is shown by way of illustration in FIG. 1, more typically a medical imaging laboratory will have multiple image acquisition devices, which may be of the same and/or different imaging modalities. For example, if a hospital performs many CT imaging examinations and relatively fewer MRI examinations and still fewer PET examinations, then the hospital's imaging laboratory (sometimes called the “radiology lab” or some other similar nomenclature) may have three CT scanners, two MRI scanners, and only a single PET scanner. This is merely an example. Moreover, the remote service center 4 may provide service to multiple hospitals. The local operator controls the medical imaging device 2 via an imaging device controller 10. The remote operator is stationed at a remote electronic processing device 12 (or, more generally, an electronic controller 12).

To provide for optional contrast-enhanced imaging, an optional contrast injector 11 is configured to inject the patient with a contrast agent. The contrast injector 11 is a configurable automated contrast injector having a display 13. The user (usually the imaging technologist) loads a vial or syringe of contrast agent (or two, or more, vials of different contrast agent components) into the contrast injector 11, and configures the contrast injector 11 by entering contrast injector settings such as flow rates, volumes, time delays, injection time durations, and/or so forth via a user interface (UI) of the contrast injector 11. The UI may be a touch-sensitive overlay of the display 13, and/or physical buttons, keypad, and/or so forth. In a variant embodiment, the contrast injector 11 is integrated with the imaging device controller 10 (e.g., via a wired or wireless data connection), and the contrast injector 11 is controlled via the imaging device controller 10, including displaying the contrast injector settings in a (optionally selectable) window on the display of the imaging device controller 10.

As used herein, the term “medical imaging device bay” (and variants thereof) refer to a room containing the medical imaging device 2 and also any adjacent control room containing the medical imaging device controller 10 for controlling the medical imaging device. For example, in reference to an MRI device, the medical imaging device bay 3 can include the radiofrequency (RF) shielded room containing the MRI device 2, as well as an adjacent control room housing the medical imaging device controller 10, as understood in the art of MRI devices and procedures. On the other hand, for other imaging modalities such as CT, the imaging device controller 10 may be located in the same room as the imaging device 2, so that there is no adjacent control room and the medical bay 3 is only the room containing the medical imaging device 2. In addition, while FIG. 1 shows a single medical imaging device bay 3, it will be appreciated that the remote service center 4 (and more particularly the remote electronic processing device 12) is in communication with multiple medical bays via a communication link 14, which typically comprises the Internet augmented by local area networks at the remote expert RE and local operator LO ends for electronic data communications. In addition, while FIG. 1 shows a single remote service center 4, it will be appreciated that the medical imaging device bays 3 is in communication with multiple medical bays via the communication link 14.

As diagrammatically shown in FIG. 1, in some embodiments, a camera 16 (e.g., a video camera) is arranged to acquire a video stream or feed (i.e., video) 17 of a portion of a workspace of the medical imaging device bay 3 that includes at least the area of the imaging device 2 where the local operator LO interacts with the patient, and optionally may further include the imaging device controller 10. In some embodiments, an optional microphone 15 is arranged to acquire an audio stream or feed 18 of the workspace that includes audio noises occurring within the medical imaging device bay 3 (e.g., verbal instructions by the local operator LO, questions from the patient, and so forth). The video 17 and/or the audio 18 is sent to the remote electronic processing device 12 via the communication link 14, e.g. as a streaming video feed received via a secure Internet link.

The communication link 14 also provides a natural language communication pathway 19 for verbal and/or textual communication between the local operator and the remote operator. For example, the natural language communication link 19 may be a Voice-Over-Internet-Protocol (VOIP) telephonic connection, an online video chat link, a computerized instant messaging service, or so forth. Alternatively, the natural language communication pathway 19 may be provided by a dedicated communication link that is separate from the communication link 14 providing the data communications 17, 18, e.g. the natural language communication pathway 19 may be provided via a landline telephone. In some embodiments, the natural language communication link 19 allows a local operator LO to call a selected remote expert RE. The call, as used herein, can refer to an audio call (e.g., a telephone call), a video call (e.g., a Skype or Facetime or other screen-sharing program), or an audio-video call. In another example, the natural language communication pathway 19 may be provided via an ROCC device 8, such as a mobile device (e.g., a tablet computer or a smartphone), or can be a wearable device worn by the local operator LO, such as an augmented reality (AR) display device (e.g., AR goggles), a projector device, a heads-up display (HUD) device, etc., each of which having a display device 36. For example, an “app” can run on the ROCC device 8 (operable by the local operator LO) and the remote electronic processing device 12 (operable by the remote expert RE) to allow communication (e.g., audio chats, video chats, and so forth) between the local operator and the remote expert.

FIG. 1 also shows, in the remote service center 4 including the remote electronic processing device 12, such as a workstation, a workstation computer, or more generally a computer, which is operatively connected to receive and present the video feed 17 of the medical imaging device bay 3 from the camera 16 and/or to the audio feed 18. Additionally or alternatively, the remote electronic processing device 12 can be embodied as a server computer or a plurality of server computers, e.g. interconnected to form a server cluster, cloud computing resource, or so forth. The electronic processing device 12 includes typical components, such as an electronic processor 20 (e.g., a microprocessor), at least one user input device (e.g., a mouse, a keyboard, a trackball, and/or the like) 22, and at least one display device 24 (e.g. an LCD display, plasma display, cathode ray tube display, and/or so forth). In some embodiments, the display device 24 can be a separate component from the electronic processing device 12. The display device 24 may also comprise two or more display devices. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the electronic processing device 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the remote operator display device 24. The video feed 17 from the camera 16 can also be displayed on the display device 24, and the audio feed 18 can be output on the remote electronic processing device 12 via a loudspeaker 29. In some examples, the audio feed 18 can be an audio component of an audio/video feed (such as, for example, recording as a video cassette recorder (VCR) device would operate).

FIG. 1 shows an illustrative local operator LO, and an illustrative remote expert RE (e.g., supertech). However, in a Radiology Operations Command Center (ROCC) as contemplated herein, the ROCC provides a staff of supertechs who are available to assist local operators LO at different hospitals, radiology labs, or the like. Each remote expert RE can operate a corresponding remote electronic processing device 12. The ROCC may be housed in a single physical location, or may be geographically distributed. For example, in one contemplated implementation, the remote expert RE are recruited from across the United States and/or internationally in order to provide a staff of supertechs with a wide range of expertise in various imaging modalities and in various imaging procedures targeting various imaged anatomies. A server computer 14s can be in communication with the medical imaging bay 3 and the remote service center 4 with one or more non-transitory storage media 26s. The non-transitory storage media 26s may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the server computer 14s, various combinations thereof, or so forth. It is to be understood that any reference to a non-transitory medium or media 26s herein is to be broadly construed as encompassing a single medium or multiple media of the same or different types. Likewise, the server computer 14s may be embodied as a single electronic processor or as two or more electronic processors. The non-transitory storage media 26s stores instructions executable by the server computer 14s.

The medical imaging device controller 10 in the medical imaging device bay 3 also includes similar components as the remote electronic processing device 12 disposed in the remote service center 4. Except as otherwise indicated herein, features of the medical imaging device controller 10, which includes a local electronic processing device 12′, disposed in the medical imaging device bay 3 similar to those of the remote electronic processing device 12 disposed in the remote service center 4 have a common reference number followed by a “prime” symbol, and the description of the components of the medical imaging device controller 10 will not be repeated. In particular, the medical imaging device controller 10 is configured to display a GUI 28′ on a display device or controller display 24′ that presents information pertaining to the control of the medical imaging device 2, such as configuration displays for adjusting configuration settings an alert 30 perceptible at the remote location when the status information on the medical imaging examination satisfies an alert criterion of the imaging device 2, imaging acquisition monitoring information, presentation of acquired medical images, and so forth. It will be appreciated that the screen mirroring data stream 18 carries the content presented on the display device 24′ of the medical imaging device controller 10. The communication link 14 allows for screen sharing between the display device 24 in the remote service center 4 and the display device 24′ in the medical imaging device bay 3. The GUI 28′ includes one or more dialog screens, including, for example, an examination/scan selection dialog screen, a scan settings dialog screen, an acquisition monitoring dialog screen, among others. The GUI 28′ can be included in the video feed 17 and displayed on the remote electronic processing device display 24 at the remote location 4.

Furthermore, as disclosed herein, the server 14s performs a method or process 100 for providing assistance during a medical imaging examination performed using a medical imaging device 2 (i.e., by assisting local operators LO of respective medical imaging devices 2 during medical imaging examinations by a remote expert RE). The instructions to perform the method 100 are stored in the non-transitory computer readable medium 26 of the remote electronic processing device 12.

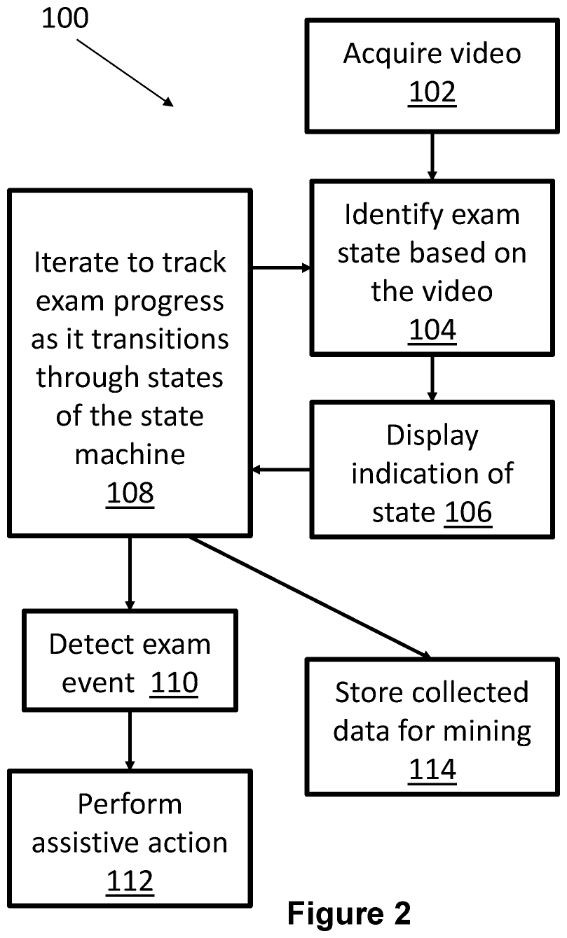

With reference to FIG. 2, and with continuing reference to FIG. 1, an illustrative embodiment of the method 100 is diagrammatically shown as a flowchart. To begin the method 100, a medical imaging examination is commenced by the local operator LO using the medical imaging device 2. An event can occur during the examination which requires assistance from a remote expert RE. While a single medical imaging examination is described, it will be appreciated that an instance of the examination progress tracking method 100 can in general be performed for each medical imaging examination monitored by the cameras of the ROCC apparatus 1.

At an operation 102, the video 17 (acquired by the one or more cameras 16) of the medical imaging examination is acquired and routed to the server computer 14s for analysis. At an operation 104, a state of the one or more imaging examinations is determined from the acquired video 17 (and optionally also the audio feed 18) using a state machine 40 implemented in the server computer 14s. At an operation 106, an indication 46 of the determined state of the one or more imaging examinations is displayed on the display device 36 of the ROCC device 8. At an operation 108 flow loops back so that the operations 104 and 106 are iterated to track progress of the medical imaging examination as it transitions through states of the state machine. At any time during the imaging examination, the tracking of the imaging examination progress may result in an operation 110 at which an examination event is detected that may call for an assistive action, in which case at an operation 112 the assistive action is performed. For example, the assistive action may include establishing the natural communication pathway 19 between the local operator LO performing the medical imaging examination and the remote expert RE. Establishing this communication may also include providing an indication to the remote expert RE of the detected event, so that the remote expert is given situational awareness of the event in the imaging examination. In another example, the assistive action may include automatically providing textual, graphical, video, and/or multimedia guidance to the local operator LO respective to the determined event. For example, if the detected event is that the state of the imaging examination transitions from image data acquisition to patient positioning (which is a regressive step), then this can trigger presentation to the user on a locally situated display of textual, graphical, video, and/or multimedia guidance on how to position the patient for the specific imaging sequence being performed. Advantageously, this latter assistive action can facilitate the local operator LO resolving the event without drawing on the valuable time of the remote expert RE. Additionally or alternatively, at an operation 114 the collected data on the progress of the examination from the iterative tracking 108 can be stored for later data mining.

The state machine 40 can comprise a plurality of states of an imaging examination. To determine a current state of an imaging examination performed by the local operator LO, an initial state of the imaging examination is determined. The indication 46 displayed on the ROCC device 8 can comprise the initial state, and the initial state can comprise patient information and imaging examination information input by the local operator LO to the ROCC device 8. A transition to a subsequent state of the imaging examination from the determined initial state can be identified using the state machine 40. When such transitions occur, the display device 36 of the ROCC device 8 can be updated to display the indication 46 as a transition of the previous state of the imaging examination to an updated state of the imaging examination.

In some embodiments, an expected duration of each state of the imaging examination can be determined, and an alert 30 indicative an alert indicative one of the states of the imaging examination exceeding the corresponding expected duration can be output via the ROCC device 8.

In some embodiments, a performance of the local operator LO performing the imaging examination can be monitored, and this performance data can be stored in the server computer 14s.

In some embodiments, the indication 46 of the determined state of the imaging examination can be displayed on the remote electronic processing device 12. In addition, the alert 30 indicative one of the states of the one or more imaging examinations exceeding a corresponding expected duration of can be output via the remote electronic processing device 12. In another example, the natural communication pathway 19 between the local operator LO and the remote expert RE can be established based on the determined state of the imaging examination (e.g., whether the local operator LO needs assistance from the remote expert RE, whether an alert 30 is output, and so forth).

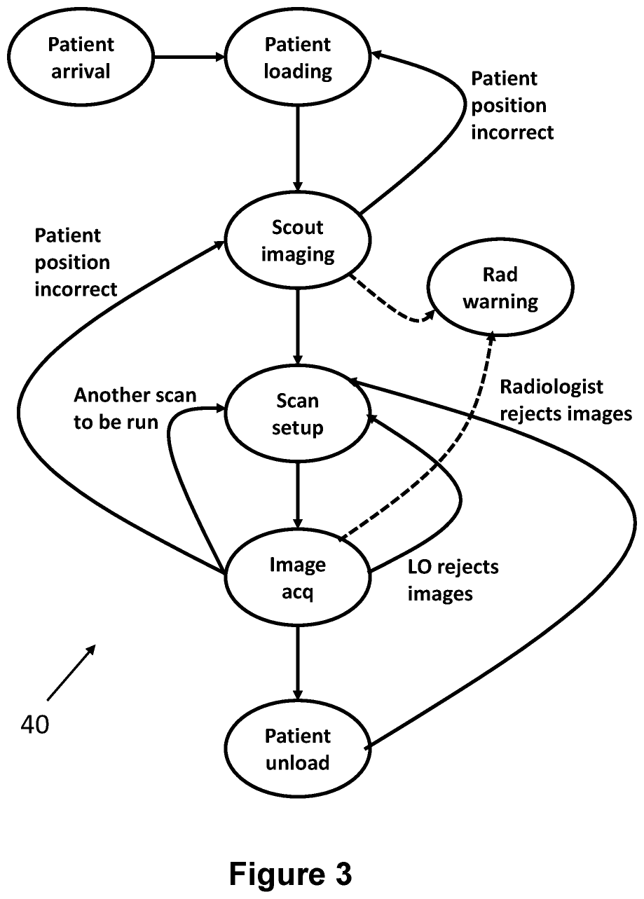

With reference to FIG. 3, an illustrative simplified state machine 40 representing a medical imaging examination workflow is diagrammatically shown. The illustrative state machine 40 includes the states of “Patient arrival”, “Patient loading”, “Scout imaging”, “Scan setup”, “imaging data acquisition”, and “Patient unloading”. The state machine 40 also includes state transitions represented by directed arrows connecting states, where the direction of the arrow is from a current state to a next state. The expected workflow is indicated by state transitions indicated by straight arrows: these include: a transition from “Patient arrival” to “Patient loading;” a transition from “Patient loading” to “Scout imaging;” a transition from “Scout imaging” to “Scan setup;” a transition from “Scan setup” to “imaging data acquisition;” and a transition from ““imaging data acquisition” to “Patient unloading.” In addition to this linear workflow, a further commonly expected transition may occur from “Imaging data acquisition” to “Scan setup,” representing the transition from one scheduled scan to a next scheduled scan. These transitions are expected and hence generally do not constitute events of the medical imaging examination that might call for assistive action (e.g. per operations 110 and 112 of FIG. 2).

On the other hand, other possible state transitions may constitute events that call for assistive action. For example, a transition from “Scout imaging” to “Patient loading” may indicate an event in which the patient was incorrectly positioned. A transition from “Image acquisition” to “Scan setup” may indicate the local operator LO has rejected the clinical images and is adjusting the scan setup for a re-scan. A transition from “Patient unloading” to “Scan setup” (or to “Patient loading,” though this transition is not shown in FIG. 3) may indicate that a reviewing radiologist has rejected the images. Such state transitions are “regressive” in the sense that the workflow is not progressing as expected and some sort of remedial action is being performed. Hence, these state transitions may trigger the operation 110 which detects an examination event on the basis of the detected regressive state transition, leading to operation 112 performing an assistive action.

In some embodiments, the next state by itself may trigger the operations 110 and 112, regardless of what transition led to that state. FIG. 3 shows an example as the additional state of “Radiation warning” which may be reached by a transition from either “Scout imaging” or “Image acquisition”. (Note, FIG. 3 depicts these transitions using dashed arrow lines). The “Radiation warning” state could arise during imaging employing ionizing radiation, such as CT, due to the calculated radiation dose delivered to the patient (calculated for example based on the X-ray tube current and duration of exposure) exceeding a predefined safety limit. Regardless of how the “Radiation warning” state is reached, it will typically trigger operations 110 and 112 so as to perform some assistive action.

The detection of an event in operation 110 may also depend on other information. For example, a single instance (or even perhaps two or three repetitions) of the transition from “Scout imaging” to “Patient loading” may not trigger an event in operation 110, since it may be typical for the local operator LO to need to iteratively position the patient, perform scout scanning, and reposition the patient to achieve optimal patient positioning. However, the operation 110 may detect an event if there are more than some threshold N number of instances of the transition from “Scout imaging” to “Patient loading”, as this excessive number of repetitions may indicate the local operator LO is having difficulty positioning the patient. Likewise, although the “scout imaging” state is a normal state of the workflow, if the medical imaging examination remains in the “Scout imaging” state for longer than an expected duration (e.g., expected based on how long it usually takes for a local operator to perform the scout scanning), then this may trigger an event in the operation 110 as it suggests the local operator LO is having some difficulty. Again, these are merely further nonlimiting illustrative examples.

It will also be appreciated that the state diagram 40 of FIG. 3 is highly simplified, and that a state machine representing an actual medical imaging examination workflow is likely to include more states and state transitions. As nonlimiting examples, the “Patient loading” state may in practice be decomposed into multiple states such as “Patient loading onto patient support,” “Patient positioning on patient support,” “Raising patient support to bore level,” “Patient support translation into scanner bore,” and/or so forth, with various transitions possible between these states. The “Scan setup” state may likewise be decomposed into various states representing stages of the scan setup process, and so forth. Various imaging modality-specific or imaging examination-specific states may also exist, such as a state representing the initiation of a contrast agent bolus delivery.

Various approaches can be used to track progress of the medical imaging examination using the state machine 40. For example, detection of a transition from a current state to a next state may involve: detecting the transition as a change in content of the acquired video 17 from a first user interface (UI) dialog screen corresponding to the current state in the state machine 40 (for example, a scout imaging UI dialog corresponding to the “Scout imaging” state) to a second UI dialog screen (which is different from the first UI dialog screen); and determining the next state of the medical imaging examination by matching the second UI dialog screen with the next state in the state machine 40 (for example, matching the second UI screen with a “scan setup” UI screen corresponding to the “Scan setup” state). In another example, detection of a transition from a current state to a next state may involve determining a change in the content of the acquired video 17; and detecting the transition of the medical imaging examination from the current state of the medical imaging examination to the next state of the medical imaging examination based on the detected change in the content of the acquired video 17 and the permissible transitions out of the current state in the state machine 40. These are merely nonlimiting illustrative examples.

### EXAMPLE

The following describes some further embodiments of the apparatus 1 and the method 100 of FIGS. 1 and 2 in more detail. The server computer 14s can comprise one or more modules configured to perform the operations of the method 100. In one example, a module for parsing the console video 17 into images is provided. This module is capable of parsing images captured from both the live video feed 17 or a recorded video at a chosen frequency.

A module for estimating changes between the images can also be provided. This module uses a reference image to identify the changes in the current image. In the current implementation, the reference image is chosen as the previous image. In other words, two consecutive images are used to detect the changes. This module also handles pre-processing of both images such as thresholding in order to detect the differences. Further, it filters the minor changes such as mouse movement. This module can be extended to classify the changes into two groups: new to the current image and removed from the current image.

A module for determining events of the imaging examination from the detected changes between images can be provided. This module analyses the detected changes and classifies the changes into pop-up windows. It uses a combination of image processing techniques with machine learning methods for this analysis. This module can also be extended to use templates for recognizing the events that have previously occurred.

A module for estimating the imaging examination context based on the determined events is also provided. There is a typical order of console states, from patient registration to pushing images to a PACS, undergone by any imaging exam. The asynchronous nature of these exam states makes it challenging to derive the exam context. This module uses the console events along with the state machine 40 for robust estimation of the current state.

This module can be extended in a way such that inputs from other sensors such as camera can be used in estimating the exam state. In addition to this, it is also responsible for pushing these events on the server computer 14s along with the respective timestamps. This determination of exam context can be implemented by using the state machine 40 as follows. In general, the state will be given by a multi-dimensional state vector:

$$S = ( {s}_{1} , {s}_{2} , … , {s}_{N} ) ,$$

where N is the number of independent or partly independent state variables. For example, a state variable s₁ could represent the exam state as one of [“not started,” “survey acquisition,” “geometry planning,” “image acquisition,” “finished” ], while a state variable S₂ could represent the screen display mode as one of [“review mode,” “scanning mode,” “planning mode” ].

The state machine 40 manages the transition between state vectors S→S′ based on the determined imaging examination events received. For each event e received, the new state variables are determined by a rule set R depending on the event and on the current state of all state variables according to

$${s}_{i} \to {s}_{i}^{′} = {R}_{i} ( e , S ) .$$

In the disclosed apparatus 1, there are multiple pipelines such as optical character recognition (OCR) that are resource intensive to extract information form console screen 10. The computationally lightweight feature of the proposed method allows it to be implemented at a higher frequency than the resource heavy pipelines and the insights derived by this method can help in optimal use of resource heavy pipelines. For example, the OCR need not be run if the screen is idle for a long time or if the screen underwent changes in a particular region, OCR is run only on that portion. The exam context can be used to trigger the OCR pipeline as follows: For each state transition S→S′, a filter

$$F ( S , {S}^{′} ) = \{ \begin{matrix}1 & if pipeline run required \\ 0 & otherwise\end{matrix}$$

determines whether an immediate pipeline run should be triggered. Independent of the state changes, the pipeline will also be triggered by a timer at regular intervals.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium having stored instructions for determining a need for assistance during a medical imaging examination, the instructions, when executed by at least one processor, cause the at least one processor to:
acquire video of the medical imaging examination;
determine, using a state machine, a current state of the medical imaging examination from the acquired video;
display an indication of the determined current state of the medical imaging examination; and
identify a transition from the current state of the medical imaging examination to a next state of the medical imaging examination based on the acquired video and the state machine, wherein the transition indicates the need for assistance during the medical imaging examination.

2. (canceled)

3. The non-transitory computer readable medium of claim 12, wherein, to identify the transition, the instructions, when executed by the at least one processor, further cause the at least one processor to:
detect the transition as a change in content of the acquired video from a first user interface (UI) dialog screen corresponding to the current state in the state machine to a second UI dialog screen; and
determine the next state of the medical imaging examination by matching the second UI dialog screen with the next state in the state machine.

4. The non-transitory computer readable medium of claim 1, wherein, to identify the transition, the instructions, when executed by the at least one processor, further cause the at least one processor to:
determine a change in content of the acquired video; and
detect the transition of the medical imaging examination from the current state of the medical imaging examination to the next state of the medical imaging examination based on the determined change in the content of the acquired video and permissible transitions out of the current state in the state machine.

5. The non-transitory computer readable medium of claim 1, wherein the instructions, when executed by the at least one processor, further cause the at least one processor to:
determine an event of the medical imaging examination that triggered the transition from the current state of the medical imaging examination to the next state of the medical imaging examination based on a state transition of the state machine from the current state to the next state.

6. The non-transitory computer readable medium of claim 5, wherein the instructions, when executed by the at least one processor, further cause the at least one processor to:
perform an assistive action to provide assistance during the medical imaging examination based on the determined event.

7. The non-transitory computer readable medium of claim 2, wherein, to perform the assistive action, the instructions, when executed by the at least one processor, further cause the at least one processor to establish a natural communication pathway between a local operator performing the medical imaging examination and a remote expert.

8. The non-transitory computer readable medium of claim 6, wherein, to perform the assistive action, the instructions, when executed by the at least one processor, further cause the at least one processor to automatically provide at least one of textual, graphical, video, or multimedia guidance respective to the determined event.

9. The non-transitory computer readable medium of claim 2, wherein the current state of the medical imaging examination is defined by state variables and the instructions, when executed by the at least one processor, further cause the at least one processor to determine values for the state variables based on information determined from the acquired video including at least patient information and imaging examination information input by a local operator.

10. The non-transitory computer readable medium of claim 1, wherein the instructions, when executed by the at least one processor, further cause the at least one processor to:
output an alert indicative of a duration of the current state of the medical imaging examination exceeding an expected duration of the current state of the medical imaging examination.

11. The non-transitory computer readable medium of claim 1, wherein the instructions, when executed by the at least one processor, further cause the at least one processor to:
collect data on a performance of a local operator performing the medical imaging examination by detecting transitions of the medical imaging examination through states of the state machine by iterative repetition; and
store the collected data related to performance of the local operator.

12. The non-transitory computer readable medium of claim 1, wherein, to display the indication, the instructions, when executed by the at least one processor, further cause the at least one processor to:
display the indication of the determined current state of the medical imaging examination on a remote processing device operable by a remote expert.

13. The non-transitory computer readable medium of claim 1, wherein the instructions, when executed by the at least one processor, further cause the at least one processor to:
establish a natural communication pathway between a local operator performing the medical imaging examination and a remote expert based on the determined current state of the medical imaging examination.

14. A method for determining a need for assistance during a medical imaging examination, the method comprising:
acquiring video of the medical imaging examination;
determining, using a state machine, a current state of the medical imaging examination from the acquired video;
displaying an indication of the determined current state of the medical imaging examination; and
identifying a transition from the current state of the medical imaging examination to a next state of the medical imaging examination based on the acquired video and the state machine, wherein the transition indicates the need for assistance during the medical imaging examination.

15. The method of 14, wherein the tracking progress includes:
determining a current state of the medical imaging examination based on a user interface (UI) screen detected in the acquired video of the medical imaging examination that corresponds to a UI screen associated with the current state in the state machine;
detecting a transition of the medical imaging examination from the current state of the medical imaging examination by detecting a change in the acquired video feed from the first UI dialog screen to a second UI dialog screen; and
determining a next state of the medical imaging examination based on matching the second UI dialog screen with a UI screen associated with a next state in the state machine for which the state machine has a transition from the current state to the next state.

16. The method of 14, wherein the tracking progress includes:
determining a change in content of the acquired video; and
detecting a transition of the medical imaging examination from a current state of the medical imaging examination to a next state of the medical imaging examination based on the detected change in the content of the acquired video.

17. The method of claim 15, further comprising:
tracking progress of the medical imaging examination using the state machine, wherein the progress is tracked based on matching information extracted from the acquired video to state information of states of the state machine;
determining an assistive action based on at least one of the determined next state or the detected transition and based on the tracked progress of the medical imaging examination; and
performing the assistive action to provide assistance during the medical imaging examination.

18. The method of claim 17, wherein the assistive action includes establishing a natural communication pathway between a local operator performing the medical imaging examination and a remote expert.

19. The method of claim 17, wherein the assistive action includes automatically providing at least one of textual, graphical, video, or multimedia guidance respective to a determined event of the medical imaging examination.

20. A system for determining a need for assistance during a medical imaging examination the system comprising:
at least one processor operatively coupled to memory, the at least one processor configured to:
acquire video of the medical imaging examination;
determine, using a state machine, a current state of an imaging examination from the acquired video;
display an indication of the determined current state of the imaging examination; and
identify a transition from the current state of the medical imaging examination to a next state of the medical imaging examination based on the acquired video and the state machine, wherein the transition indicates the need for assistance during the medical imaging examination.

21. The system of claim 1, wherein the at least one processor is further configured to:
detect the transition as a change in content of the acquired video from a first UI dialog screen corresponding to the current state in the state machine to a second UI dialog screen; and
determine the next state of the medical imaging examination by matching the second UI dialog screen with the next state in the state machine.
